(19) United States
(12) Patent Application Publication
(10) Pub. No.: US 2025/0122463 A1
(43) Pub. Date: Apr. 17, 2025

(54) BIOREACTOR FOR NON-CONTACT SENSING

(71) Applicant: Caladan Bio Inc, Brooklyn, NY (US)

(72) Inventors: Michael Kobida, JR., New York, NY (US); Robert Jauquet, Jersey City, NJ (US)

(21) Appl. No.: 18/913,706

(22) Filed: Oct. 11, 2024

Related U.S. Application Data
(60) Provisional application No. 63/543,678, filed on Oct. 11, 2023.

(51) Int. Cl. C12M 1/00; C12M 1/06; C12M 1/34

(52) U.S. Cl. CPC C12M 41/08; C12M 23/22; C12M 23/58; C12M 27/06; C12M 29/18; C12M 41/26; C12M 41/34; C12M 41/44

(57) ABSTRACT

A bioreactor system includes a reactor tank, a liquid input line in fluid communication with the reactor tank to provide a liquid medium to an interior of the reactor tank, a gas input line in fluid communication with the reactor tank to provide a gas to the interior of the reactor tank, and an agitator positioned in the reactor tank. The bioreactor system includes a sight glass disposed on a wall of the reactor tank. The sight glass is positioned at a first height from a bottom of the reactor tank. The first height lower is than a second height indicative of a maximum fill height for the liquid medium within the reactor tank. The sight glass is adapted to interact with light-based measurement technologies.

### CROSS-REFERENCE TO RELATED APPLICATIONS

This application claims priority from U.S. Provisional Application No. 63/543,678, filed Oct. 11, 2023, and titled “RAMAN SPECTROMETRY IN BIOREACTOR SYSTEMS”, the entire disclosure of which is incorporated by reference for all purposes.

### TECHNICAL FIELD

This application relates generally to bioreactor vessels and more particularly to monitoring and sensing conditions within a bioreactor vessel using non-contact sensing.

### BACKGROUND

A bioreactor refers to a device that supports the cultivation and processing of biological materials, such as cells, microorganisms, and biologically derived products. Typically, a bioreactor is a vessel in which a chemical process is carried out which involves organisms or biochemically active substances derived from such organisms. Cell cultures, consisting of cells growing suspended in a growth media, or on the surface of suspended particles, in solution, are produced within bioreactors with careful control of several parameters. Within a bioreactor, it is important to carefully control the environment to which the cells are exposed. Subtle changes in the environment may have major effects on the physiology of the cells and the amount of the target product. This in turn has a major impact on the economics of the production process. This process may either be aerobic or anaerobic. These bioreactors are commonly cylindrical, ranging in size from liters to cubic meters.

Bioreactors are vital tools in various industries, including biotechnology, pharmaceuticals, and industrial microbiology. Bioreactors are essential for the controlled cultivation and production of a wide array of biological materials, such as therapeutic proteins, vaccines, antibiotics, and biofuels. Single-use bioreactors have gained popularity due to their advantages, such as reduced contamination risk and simplified maintenance; however, traditional single-use bioreactors face several limitations, particularly concerning real-time monitoring and measurement capabilities. Existing bioreactor systems are frequently limited by their inability to collect continuous data throughout experiments, thus impeding the ability to make precise adjustments and enhance processes effectively. Conventional single-use bioreactors often lack the necessary interfaces for advanced measurement techniques, such as Raman spectroscopy, which allows for in-situ analysis of the composition and quality of biological media during the bioprocessing. By integrating Raman spectrometry into bioreactors, real-time monitoring of critical parameters like glucose levels, cell density, and amino acid concentrations becomes possible. Raman spectroscopy refers to a spectroscopic technique used to determine vibrational modes of molecules. Raman spectroscopy is used to provide a structural fingerprint by which molecules may be identified. Raman spectroscopy relies upon inelastic scattering of photons, known as Raman scattering. A source of monochromatic light, usually from a laser in the visible, near infrared or near ultraviolet range is used. The laser light interacts with molecular vibrations, phonons or other excitations in the system, resulting in the energy of the laser photons being shifted up or down. The shift in energy gives information about the vibrational modes in the system.

Typically, a sample is illuminated with a laser beam. Electromagnetic radiation from the illuminated spot is collected with a lens and sent through a monochromator. Elastic scattered radiation at the wavelength corresponding to the laser line (Rayleigh scattering) is filtered out by either a notch filter, edge pass filter or a band pass filter, while the rest of the collected light is dispersed onto a detector. However, the prohibitive costs and complex installation requirements of individual Raman spectrometers have discouraged their widespread use in existing bioreactor systems. Furthermore, the materials commonly used in single-use bioreactors may not be suitable for Raman spectroscopy due to their inherent lack of transparency and inconsistent Raman signatures.

Therefore, there is a need for cost effective bioreactors and systems to integrate Raman spectrometry into bioreactor systems to enable real-time monitoring of critical parameters.

### SUMMARY

Various implementations of the present disclosure relate to a bioreactor including a reactor tank, a liquid input line in fluid communication with the reactor tank to provide a liquid medium to an interior of the reactor tank, a gas input line in fluid communication with the reactor tank to provide a gas to the interior of the reactor tank, and an agitator positioned in the reactor tank. The bioreactor may include a sight glass disposed on a wall of the reactor tank. The sight glass may be positioned at a first height from a bottom of the reactor tank. The first height may be lower than a second height indicative of a maximum fill height for the liquid medium within the reactor tank, and the sight glass may be adapted to interact with light-based measurement technologies.

In some examples, the sight glass may include a planar and optically transparent material. The sight glass may include sapphire or borosilicate glass. In some examples, the wall of the reactor tank may include a curved wall portion defining an opening and the sight glass includes a planar material secured to the curved wall portion at the opening, the sight glass secured by a frame connecting the planar material to the curved wall portion. In some examples, the sight glass is positioned perpendicular to a radius of the reactor tank. In some examples, the reactor tank tapers from a first diameter adjacent a top end to a second diameter adjacent a bottom end, the first diameter greater than the second diameter, and wherein the sight glass is disposed vertically and perpendicular to an incident ray of the light-based measurement technologies. In some examples, the light-based measurement technologies include a Raman spectroscopy probe.

In some examples, the systems described herein may relate to one or more bioreactors, each bioreactor of the one or more bioreactors including a reactor tank and a sight glass disposed on a wall of the reactor tank, the sight glass positioned at a first height from a bottom of the reactor tank, the first height lower than a second height indicative of a maximum fill height for a liquid medium within the reactor tank. The system also includes an optical probe configured to perform optical spectroscopy to determine sensor data associated with an environment within the reactor tank and a conveyance device configured to position the optical probe to align with the sight glass of the one or more bioreactors. In some examples, the one or more bioreactors are arranged adjacent one another along a first axis and the conveyance device includes a track-based system configured to position the optical probe at positions along the first axis such that the optical probe aligns with respective sight glasses of the one or more bioreactors. The conveyance device is configured to maintain the optical probe perpendicular to the sight glass of the one or more bioreactors as the optical probe moves between the one or more bioreactors. In some examples, the optical probe includes an optical source configured to produce light used for spectroscopic analysis of contents of the reactor tank, and a cable optically connected to the optical source. The cable may be configured to transport the light from the optical source to an end of the cable and transport light to a spectrometer from the reactor tank. A cart may be disposed on the conveyance device and connected to the end of the cable. The cart may be further configured to move between the one or more bioreactors for performing spectroscopy of the one or more bioreactors using the optical source. In some examples, the system further includes one or more probe locating features disposed on respective bioreactors of the one or more bioreactors, the one or more probe locating features configured to guide positioning of the optical probe between the one or more bioreactors. In some examples, the optical probe includes a laser source, a spectrometer, and a fiber optic cable connected between the laser source, the spectrometer, and the conveyance device. The fiber optic cable may be configured to carry first light from the laser source to the reactor tank and carry second light to the spectrometer from the reactor tank.

### BRIEF DESCRIPTION OF THE DRAWINGS

The following figures, which form a part of this disclosure, are illustrative of described technology and are not meant to limit the scope of the claims in any manner.

FIG. 1 illustrates an example of a bioreactor system using a multi-channel peristaltic pump to deliver liquid into a bioreactor chamber, according to an embodiment of this disclosure.

FIG. 2 illustrates a section view of the bioreactor system of FIG. 1, according to an embodiment of this disclosure.

FIG. 3 illustrates a perspective view of the bioreactor system of FIG. 1, according to an embodiment of this disclosure.

FIG. 4 illustrates a reactor tank for a bioreactor system including a sight window, according to an embodiment of this disclosure.

FIG. 5 illustrates a reactor tank for a bioreactor system, according to an embodiment of this disclosure.

FIG. 6 illustrates a section view of a reactor tank for a bioreactor system, according to an embodiment of this disclosure.

FIG. 7 illustrates a diagram of a bioreactor array including multiple bioreactor systems with a single optical emitter and sensor to perform optical sensing of characteristics within the bioreactor systems, according to an embodiment of this disclosure.

FIG. 8 illustrates a section view of a bioreactor system interfacing with an optical probe for spectroscopic analysis of the bioreactor tank, according to an embodiment of this disclosure.

### DETAILED DESCRIPTION

Various implementations of the present disclosure will be described in detail with reference to the drawings, wherein like reference numerals present like parts and assemblies throughout the several views. In the figures, the left-most digit(s) of a reference number identifies the figure in which the reference number first appears. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity. Additionally, any samples set forth in this specification are not intended to be limiting and merely set forth some of the many possible implementations.

A system as described herein may include a bioreactor system that includes a bioreactor vessel contained within a housing and connected through one or more tubes to a source of nutrients for providing to the interior of the bioreactor vessel. Biorcactors include a reactor tank which typically is a cylindrical container. A liquid medium may be provided by a feeding pump via a liquid input line. Liquid input lines enable precise control over the addition of nutrients, supplements or other liquids required for the bioprocess. The control of fluids is important for maintaining optimal conditions. Gas may be provided by a gas pump via a gas input line. Gas input lines allow for the controlled delivery of gases, including oxygen and carbon dioxide, which are important for cellular respiration and pH regulation in the bioreactor.

The bioreactor system may include a reactor tank made from a material such as a metal or polycarbonate, which is not a suitable material for optical sensing, such as spectroscopy for monitoring and sensor determination of the contents of the reactor tank. For example, polycarbonate or metal reactor tanks may prevent a laser for a spectroscopy to sense through. In accordance with the principles of the present disclosure, a sight glass is provided on the reactor tank. A durable rubber seal may be placed between the sight glass and the reactor tank to establish a watertight seal, preventing any leakage or contamination. The sight glass material is substantially transparent, allowing laser waves from spectrometer, such as a Raman spectrometer to penetrate it and interact with the biological media inside the vessel. Raman spectroscopy includes using a source of monochromatic light, such as from a laser, to interact with molecular vibrations or other excitations in a system that results in the energy of the laser photons being shifted. The shift in energy provides information about vibrational modes at the sample. The returned, reflected light is detected by a detector and the detected light (with shifted photons) yields information related to the chemical structure, crystallinity, molecular dynamics, and polymorphism of the sample. Accordingly, it is important that the reactor tank be transparent, at least in part to enable in-situ spectroscopy measurements. Though described in some example with respect to Raman spectroscopy, other spectroscopic methods may also be used such as infrared spectroscopy or other such optical techniques for gathering information related to the contents of the reactor tank. In addition to transparency, the laser needs to penetrate undisturbed, or primarily undisturbed, into the interior of the reactor tank. Accordingly, a sight glass is incorporated into the reactor tank to enable high optical clarity through the sight glass to the interior of the reactor tank. This sight glass enables the remainder of the tank to be formed of a material such as polycarbonate or a metal such as stainless steel.

The sight glass is made of a material that is suitable for use with Raman spectroscopy or other light-based measurement technologies. A suitable material is one which is transparent and of a known, consistent Raman signature, making it possible for the waves of the laser to pass through the material. Additionally, the sight glass material possesses a well-documented and consistent Raman signature that is distinguishable from the Raman signatures of the biological media included within the reactor tank. In some examples the sight glass may be formed of BK7 borosilicate glass and/or sapphire, or other such materials known for their optical transparency and unique Raman spectra.

The sight glass is positioned such that it is perpendicular to an incident ray of the incident light of the laser. Accordingly, though the wall of the reactor tank may have a tapered cylindrical shape, the sight glass may be affixed to an opening in a side of the wall through a frame to seal the opening and also position the plane of the sight glass perpendicular to the incident light from the laser. The sight glass is flat and planar, and positioned perpendicular to the laser incident from the spectrometer. The sight glass may be parallel with a vertical axis of the reactor tank, the vertical axis aligned with a direction of gravity. The sight glass is positioned on the reactor tank, situated below the highest point of liquid within the reactor tank. The sight glass may be oriented perpendicular to a laser source and probe of the Raman spectrometer, ensuring an unobstructed path for measurements.

An agitator may be provided consisting of a motor driving a shaft having a plurality of mixing propellers positioned in the reactor tank. The inclusion of an agitator ensures thorough mixing of the biological media, promoting even distribution of nutrients and gasses. The agitator enhances the growth and productivity of the cultured organisms. Sensors connected to a system monitor, such as for example a pH sensor and a dissolved oxygen sensor may be provided. The pH sensor provides real-time monitoring and feedback on the acidity or alkalinity of the biological media. This information is important for maintaining the desired pH level throughout the bioprocessing. The dissolved oxygen sensor measures the concentration of oxygen dissolved in the biological media, providing important data for ensuring adequate oxygen supply to the cultured organisms.

The sight glass may be located in-line with one of the agitators, such as in-line with mixing propellers within the reactor tank. This placement maximizes agitation at the sight glass location, reducing the potential for film buildup on the sight glass and enhances the flow of biological media past the sight glass. This, in turn, contributes to increased homogeneity during bioprocessing, an important factor in maintaining product quality and yield as well as providing improved spectroscopy results during sampling.

In accordance with an example of the present disclosure an innovative approach to bioreactor systems is provided, enabling the continuous acquisition of data and closed-loop control through the seamless integration of spectrometry. The bioreactor systems may be arranged adjacent one another, e.g., along a first axis. Each of the bioreactor systems may be equipped with a reactor tank and each of the bioreactor systems may be equipped with a port for probe compatibility. The port is aligned with the sight glass of the reactor tank to enable the probe to capture data with respect to the internal environment of the reactor tank. In examples, the port may provide direct visibility from a probe to the sight glass. In examples, the port may include an optical material that is used to direct the laser light from the probe into the sight glass and return the reflected light to the probe. Such elements may include mirrors and optically transparent materials to reflect and transport the light as needed to reach between the sight glass and the probe. The probe is moved from one bioreactor to the next to obtain data from samples during the length of an experiment, thereby eliminating the need for multiple probes for multiple bioreactors and thereby providing cost and space-saving measures.

The length of an experiment using a bioreactor system may range significantly, such as for example from two to fourteen days. A datapoint may be collected by the probe from each bioreactor at given intervals, such as for example with each bioreactor system being measured every fifteen minutes. The duration of each data point collection will vary depending on the nature of the media inside the reactor tank, such as for example for thirty seconds to five minutes; longer sampling intervals may be strategically chosen to maximize data accuracy while considering specific media properties. In examples, the intervals between sampling and the length of time for sampling each of the bioreactor systems using the probe may be determined and/or controlled based on control information from a central controller that receives inputs related to the reactions occurring in each bioreactor and other information such as monitoring intervals, expected time periods for reactions to occur, and other such information. The central controller may adjust, in some examples, the sampling rate for the bioreactor systems based on the data obtained. For example, if a particular bioreactor system is reaching completion of a reaction sooner than the other bioreactor systems, the central controller may determine to increase the sampling rate of the particular bioreactor system until the reaction is determined to be completed or to have reached a predetermined threshold.

Throughout the length of an experiment, the probe may be precisely and rapidly moved from one bioreactor system to another at predetermined intervals as determined and/or controlled by the central controller. To facilitate the precise movement, each reactor tank and/or bioreactor system includes probe locating features. The probe locating features may include physical features such as physical stops, channels, grooves, and ridges to aid in positioning the probe. The probe locating features may be configured to guide positioning of the optical probe with respect to the bioreactor system. The probe locating features may also include visible or non-visible markers such as unique visual identifiers that may be detected by a camera system of a probe and/or conveyor device and/or non-visual features such as conductive, capacitive, and/or magnetic sensors and systems to detect alignment of the probe with the bioreactor system. The probe-locating features are important in accurately positioning the probe in relation to the specialized port. These features enable probe placement with an accuracy level, such as for example within 0.5 mm in the X and Y directions and 0.1 mm in the Z direction. The Z direction measurement defines the distance between the end of the probe and the surface of the sight glass or another port feature on the reactor tank. The placement of the probe is important for consistent measurements using the probe.

The probe is mounted to a positionable cart that may include a motorized cart that smoothly traverses along a linear rail system. Other linearly positionable systems such as conveyor systems, linear actuators, linear rails, track-based systems, and other such linearly positionable systems may be implemented to adjust a position of the probe along the axis extending the various bioreactor systems. The cart may be motorized and may precisely locate the probe with respect to the sight glass. The linearly positionable system may include a robotic system that facilitates the accurate positioning of the probe with respect to the sight glass or port of the bioreactor system, ensuring data accuracy and consistency between measurements. The three-dimensional position of the probe with respect to the sight glass may be adjusted by additional actuators to shift a position of the probe along the X, Y, or Z directions. In examples, the probe may rest on a cart that is linearly positionable along a first axis that extends across the multiple bioreactor systems. The position of the probe with respect to directions perpendicular to the first axis may be fixed, to ensure accuracy and consistency of measurement.

The probe is connected to a spectrometer and laser source. The probe may be connected to a Raman spectrometer and a laser source via a flexible cable including fiber optics. For example, the flexible cable may include two fiber optic cables, one for transmitting the laser from the laser source to be emitted at the probe and the second for carrying light from the probe to the spectrometer. The flexible cable enables the probe to move between the bioreactor systems without requiring the entire spectrometer to be movable, and instead provides for only the probe to be moved, which contributes to accuracy and consistency of positioning.

Using bioreactors and systems in accordance with the principles of the present description offers an array of advantages over current bioreactor systems and bioprocess control, including continuous data monitoring, cost-efficiency, motion precision, and enhanced efficiency. Continuous data monitoring enables real-time data acquisition which empowers scientists and operators to make precise, instantaneous adjustments to bioprocess conditions, leading to better control and process optimization. Cost-efficiency is achieved with the innovative shared use of one spectrometer across multiple bioreactors which significantly reduces integration costs, making advanced monitoring accessible to a broader range of applications. Motion precision is achieved utilizing the robotic features, which ensures consistent, accurate probe positioning, resulting in dependable measurements of cell media parameters throughout experiments. Enhanced efficiency results from automated data collection and integration which streamlines experiment management and decision-making, promoting higher productivity and resource utilization.

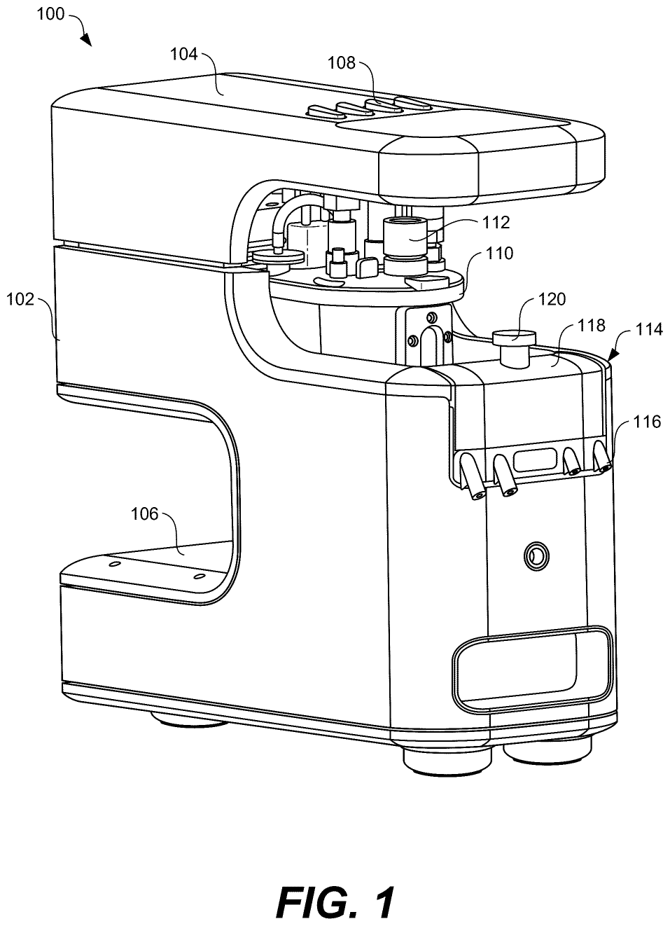

FIGS. 1-3 illustrate an example of a bioreactor system 100 that includes a bioreactor tank having a sight glass for spectrometry and enabling optical sensing through the bioreactor system. FIG. 1 illustrates an example of a bioreactor system 100 for use with optical spectroscopy, according to an example. The bioreactor system 100 may be similar or identical to a bioreactor system such as described in Provisional Patent Application No. 63/543,678, filed Oct. 11, 2023, and titled “Raman Spectrometry in Bioreactor Systems”, the disclosure of which is incorporated herein by this reference for all purposes.

A bioreactor system 100 refers to a device that supports the cultivation and processing of biological materials, such as cells, microorganisms, and biologically derived products. Typically, a bioreactor includes a vessel 110 in which a chemical process is carried out which involves organisms or biochemically active substances derived from such organisms. This process may either be aerobic or anaerobic. These bioreactor vessels are commonly cylindrical, ranging in size from liters to cubic meters.

Bioreactor systems 100 are vital tools in various industries, including biotechnology, pharmaceuticals, and industrial microbiology. Bioreactor systems 100 are essential for the controlled cultivation and production of a wide array of biological materials, such as therapeutic proteins, vaccines, antibiotics, and biofuels. Single-use bioreactor systems have gained popularity due to their advantages, such as reduced contamination risk and simplified maintenance; however, traditional single-use bioreactors face several limitations, particularly concerning real- time monitoring and measurement capabilities.

Typical bioreactor systems are frequently limited by their inability to collect continuous data throughout experiments, thus impeding the ability to make precise adjustments and enhance processes effectively. Conventional single-use bioreactor systems often lack the necessary interfaces for advanced measurement techniques, such as Raman spectroscopy, which allows for in-situ analysis of the composition and quality of biological media during the bioprocessing. By integrating Raman spectrometry into bioreactors, real-time monitoring of critical parameters like glucose levels, cell density, and amino acid concentrations become possible. Raman spectroscopy refers to a spectroscopic technique used to determine vibrational modes of molecules. Raman spectroscopy is used to provide a structural fingerprint by which molecules may be identified. Raman spectroscopy relies upon inelastic scattering of photons, known as Raman scattering. A source of monochromatic light, usually from a laser in the visible, near infrared or near ultraviolet range is used. The laser light interacts with molecular vibrations, phonons or other excitations in the system, resulting in the energy of the laser photons being shifted up or down. The shift in energy gives information about the vibrational modes in the system.

Typically, a sample is illuminated with the laser beam. Electromagnetic radiation from the illuminated spot is collected with a lens and sent through a monochromator. Elastic scattered radiation at the wavelength corresponding to the laser line (Rayleigh scattering) is filtered out by either a notch filter, edge pass filter or a band pass filter, while the rest of the collected light is dispersed onto a detector. However, the prohibitive costs and complex installation requirements of individual Raman spectrometers have discouraged their widespread use in existing bioreactor systems. Furthermore, the materials commonly used in single-use bioreactors may not be suitable for Raman spectroscopy due to their inherent lack of transparency and inconsistent Raman signatures.

The bioreactor system 100 includes a housing 102 that houses components of the system as well as a lid 104 that, when combined with the housing 102, encloses the vessel 110. The housing 102 may include and/or contain a controller, such as a computing device that may provide control of operations and devices as described herein. The housing 102 defines an imaging port 106 through which one or more optical sensors may detect characteristics or data relating to the contents of the bioreactor system 100. The imaging port 106 is shown as forming a passage into the housing 102. An additional port (not shown in FIG. 1) provides optical line of sight between a probe (e.g., Raman probe) and the sight glass of the reactor tank. One or more interfaces 108 on the lid 104 may be used to control operation of the bioreactor system 100, such as to control temperature, sample collection, nutrient delivery, timing, and other such parameters of the system contained within the bioreactor system 100.

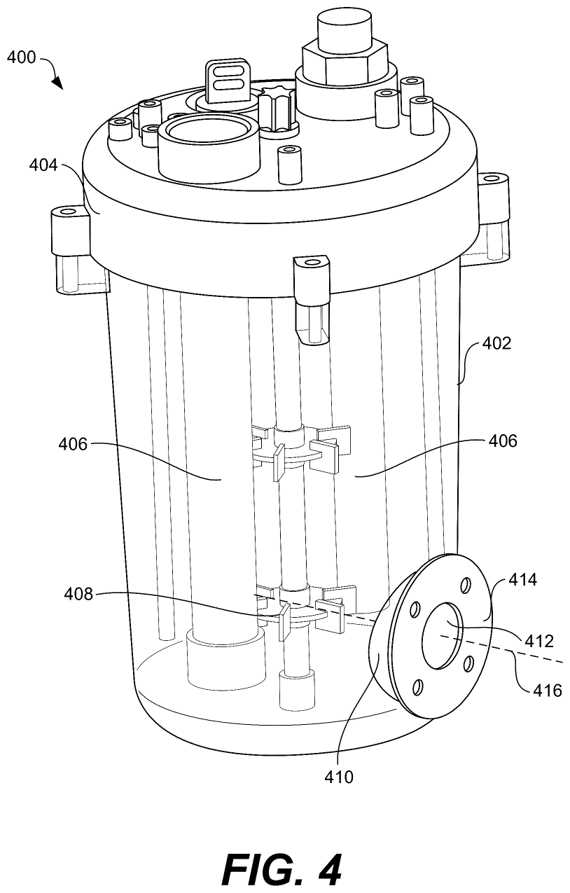

The vessel 110 includes a reactor tank which typically is a cylindrical container having a lid 112 that encloses a volume for containing and controlling an environment within the vessel 110. A liquid medium may be provided by a pump system 114 via one or more liquid input lines 116. Pumps included under cover 118 and secured in place by clip 120 may provide fluid into the vessel 110 through tubing. Liquid input lines 116 enable precise control over the addition of nutrients, supplements or other liquids required for the bioprocess within the vessel 110. This control is important for maintaining optimal conditions for a particular reaction or bioprocess. Gas may be provided by a gas pump via a gas input line. Gas input lines allow for the controlled delivery of gases, including oxygen and carbon dioxide, which are important for cellular respiration and pH regulation in the vessel 110. The vessel is shown and described in greater detail with respect to FIGS. 4-6 herein. The vessel 110 includes a sight glass (not shown in FIG. 1), as described herein, to enable a probe of a spectrometer to convey laser light into the interior of the vessel 110 and detect reflections as well through the sight glass.

The vessel 110 may be made from a material such as a metal or polycarbonate, which is not a suitable material for optical sensing, such as spectroscopy for monitoring and sensor determination of the contents of the reactor tank. For example, polycarbonate or metal vessels 110 may prevent a laser for a spectroscopy to sense through. In accordance with the principles of the present description, a sight glass is provided on the vessel 110. A durable rubber seal may be placed between the sight glass and the wall of the vessel 110 to establish a watertight seal, preventing any leakage or contamination. The sight glass material is substantially transparent, allowing laser waves from spectrometer, such as a Raman spectrometer to penetrate it and interact with the biological media inside the vessel 110. Raman spectroscopy includes using a source of monochromatic light, such as from a laser, to interact with molecular vibrations or other excitations in a system that results in the energy of the laser photons being shifted. The shift in energy provides information about vibrational modes at the sample. The returned, reflected light is detected by a detector and the detected light (with shifted photons) yields information related to the chemical structure, crystallinity, molecular dynamics, and polymorphism of the sample. Accordingly, a reactor tank that is transparent, may, at least in part, enable in-situ spectroscopy measurements. Though described in some example with respect to Raman spectroscopy, other spectroscopic methods may also be used such as infrared spectroscopy or other such optical techniques for gathering information related to the contents of the vessel 110. In addition to transparency, the laser may penetrate undisturbed, or primarily undisturbed, into the interior of the vessel 110. Accordingly, a sight glass is incorporated into the reactor tank to enable high optical clarity through the sight glass to the interior of the vessel 110. This sight glass enables the remainder of the vessel 110 to be formed of a material such as polycarbonate or a metal such as stainless steel.

The sight glass is made of a material that is suitable for use with Raman spectroscopy or other light-based measurement technologies. A suitable material is one which is transparent and of a known, consistent Raman signature, making it possible for the waves of the laser to pass through the material. Additionally, the sight glass material possesses a well-documented and consistent Raman signature that is distinguishable from the Raman signatures of the biological media included within the vessel 110. In some examples the sight glass may be formed of BK7 borosilicate glass and/or sapphire, or other such materials known for their optical transparency and unique Raman spectra.

The sight glass is positioned such that it is perpendicular to the incident light of the laser. Accordingly, though the wall of the vessel 110 may have a tapered cylindrical shape, the sight glass may be affixed to an opening in a side of the wall through a frame to seal the opening and also position the plane of the sight glass perpendicular to the incident light from the laser. The sight glass is flat and planar, and positioned perpendicular to the laser incident from the spectrometer. The sight glass may be parallel with a vertical axis of the vessel 110, the vertical axis aligned with a direction of gravity. The sight glass is positioned on the vessel 110, situated below the highest point of liquid within the reactor tank. The sight glass may be oriented perpendicular to a laser source and probe of the Raman spectrometer, ensuring an unobstructed path for measurements. The sight glass may be positioned lower than a height indicative of a maximum fill height for liquid medium within the vessel 110.

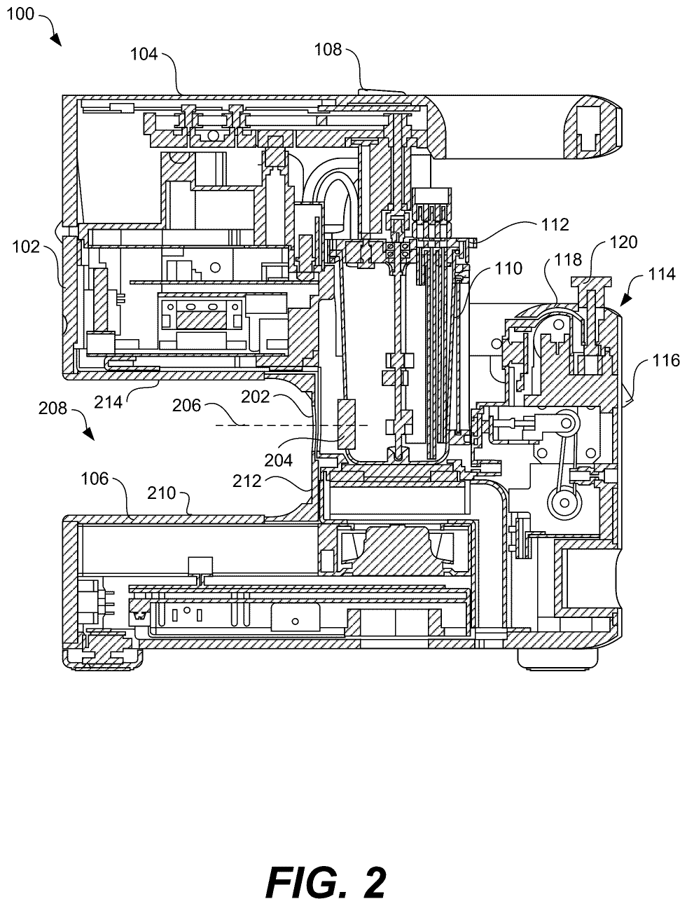

FIG. 2 illustrates a section view of the bioreactor system 100 of FIG. 1, according to an example. In the section view of the bioreactor system 100, the vessel 110 is shown surrounded by the components of the bioreactor system 100 for performing various functions such as pumping fluids through the pump system 114, heating, cooling, sensing, and otherwise interacting with the vessel 110. FIG. 2 illustrates the housing 102 of the bioreactor system 100 defining an opening 202 that provides visibility and access to a sight glass 204 of the vessel 110. An axis 206 that passes through the opening 202 and the sight glass 204 may define a sensing axis for a probe of a spectrometer to emit laser light into the vessel 110. The axis 206 is perpendicular to the sight glass 204.

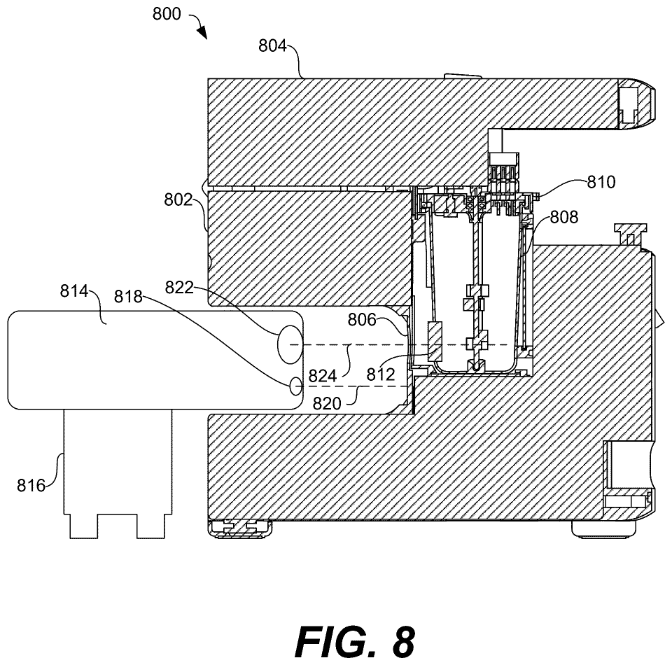

As depicted, the housing 102 defines an open portion 208 that is shaped and configured to receive a probe of the spectrometer, such as a Raman spectrometry probe. The open portion 208 is defined by a lower surface 210, side 212, and upper surface 214. The open portion 208 may be sized and shaped to receive the probe, such as shown and described with respect to FIG. 8 herein. In examples, the probe may rest on the lower surface 210 and/or contact the side 212 to index the probe against the bioreactor system 100 to ensure accuracy and consistency. This may be in addition to alignment features disposed within the bioreactor system 100.

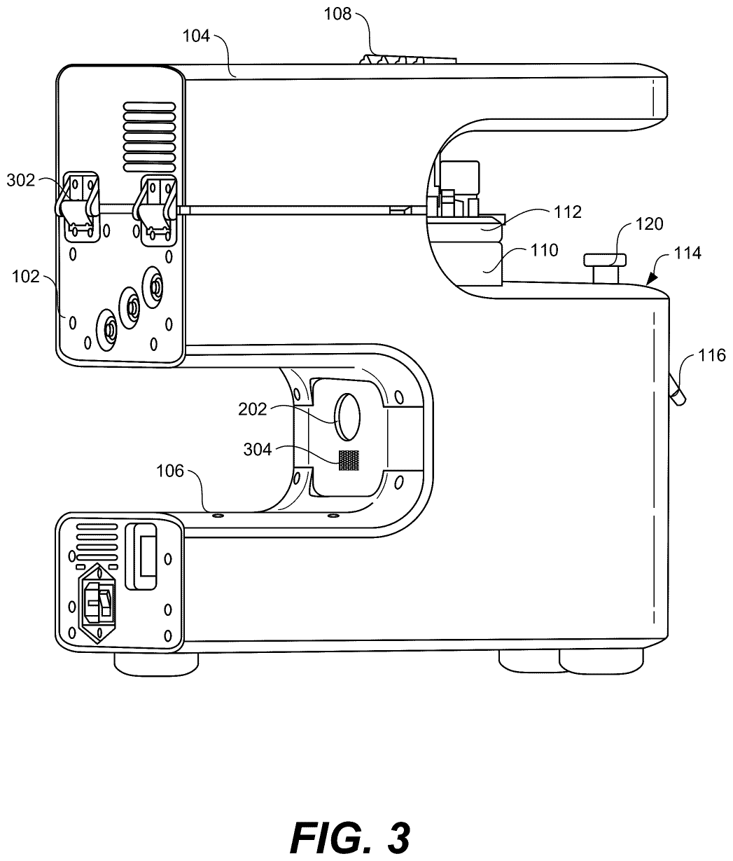

To facilitate precise movement and positioning of the probe relative to the bioreactor system 100, and in particular to the sight glass 204 of the vessel 110, each vessel 110 and/or bioreactor system 100 may include probe-locating features, such as shown in FIG. 3. In FIG. 3, the bioreactor system 100 is depicted in perspective view such that hinges 302 between the lid 104 and the housing 102 are visible, as well as connectors and ports for power and interfaces to the bioreactor system 100. Additionally, the probe locating feature 304 is shown on the side 212.

The probe locating feature 304 may include physical features such as physical stops, channels, grooves, and ridges positioned on the lower surface 210, side 212, or upper surface 214 to aid in positioning the probe. The probe locating feature 304 may also include visible or non-visible markets such as unique visual identifiers that may be positioned on or adjacent one or more of the lower surface 210, side 212, or upper surface 214 that may be detected by a camera system of a probe and/or conveyor device and/or non-visual features such as conductive, capacitive, and/or magnetic sensors and systems to detect alignment of the probe with the bioreactor system 100. The probe-locating feature 304 may be used for accurately positioning the probe in relation to the opening 202 and the sight glass 204. These features enable probe placement with an accuracy level, such as for example within 0.5 mm in the X and Y directions (perpendicular to axis 206) and 0.1 mm in the Z direction (parallel with axis 206). The Z direction measurement defines the distance between the end of the probe and the surface of the sight glass 204 or another port feature on the reactor tank. The placement of the probe is important for consistent measurements using the probe.

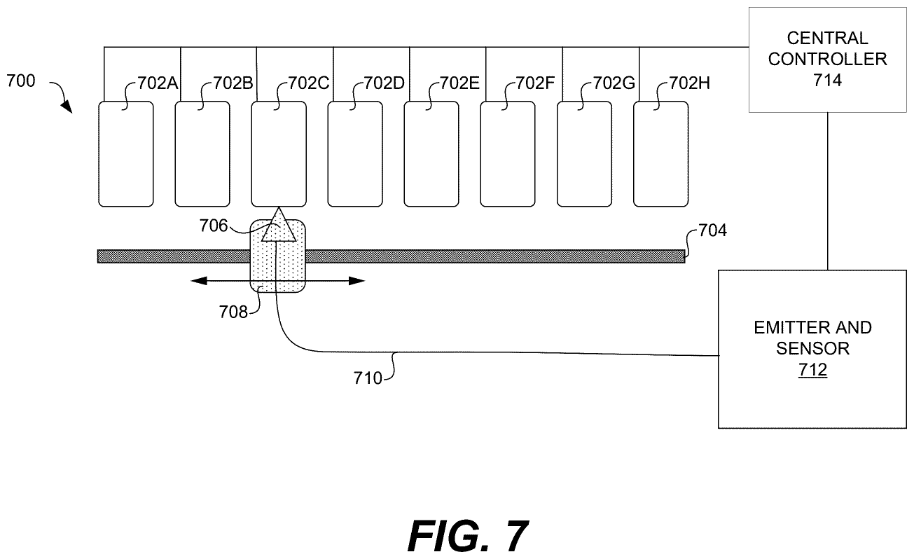

The probe is mounted to a positionable cart that may include a motorized cart that smoothly traverses along a linear rail system. Other linearly positionable systems such as conveyor systems, linear actuators, linear rails, and other such linearly positionable systems may be implemented to adjust a position of the probe into and out of the open portion 208, e.g., perpendicular to the page in FIG. 2, parallel to an axis extending between adjacent bioreactor systems, such as depicted in FIG. 7. The cart may be motorized and is able to precisely locate the probe with respect to the sight glass. The linearly positionable system may include a robotic system that facilitates the accurate positioning of the probe with respect to the sight glass or port of the bioreactor system, ensuring data accuracy and consistency between measurements. The three-dimensional position of the probe with respect to the sight glass may be adjusted by additional actuators to shift a position of the probe along the X, Y, or Z directions. In examples, the probe may rest on a cart that is linearly positionable along a first axis that extends across the multiple bioreactor systems. The position of the probe with respect to directions perpendicular to the first axis may be fixed, to ensure accuracy and consistency of measurement.

FIG. 4 illustrates a reactor tank 400 for a bioreactor system including a sight window, according to an example. The reactor tank 400 may fit within the bioreactor system 100 of FIG. 1. The reactor tank 400 includes a body 402 and a lid 404. The body 402 and the lid 404 secure together to provide an air-tight and sealed environment within the reactor tank 400. The body 402 has a generally cylindrical shape that tapers from a narrower bottom end to a wider upper end. The lid 404 includes connections for fittings such as gas, fluid, and sensors to enter into the interior of the reactor tank 400 without opening the reactor tank 400. For example, the reactor tank 400 is shown with sensors 406 that may be used to detect pH, dissolved oxygen, temperature, and other parameters associated with the interior of the reactor tank 400. Sensors 406 are connected to a system monitor and include sensors such as for example a pH sensor and a dissolved oxygen sensor. The pH sensor provides real-time monitoring and feedback on the acidity or alkalinity of the biological media. This information is important for maintaining the desired pH level throughout the bioprocessing. The dissolved oxygen sensor measures the concentration of oxygen dissolved in the biological media, providing important data for ensuring adequate oxygen supply to the cultured organisms.

The reactor tank 400 may be formed of a suitable material for containing a reaction, such as a polycarbonate, stainless steel, or other such rigid material that may be sterilized before introducing reactants.

The reactor tank 400 also includes an agitator 408 with a shaft that is driven by a motor of the bioreactor system 100. The shaft is connected to a plurality of mixing propellers positioned in the reactor tank 400. The agitator 408 may be rotated to cause stirring of the contents in the reactor tank 400 and ensures thorough mixing of the biological media, promoting even distribution of nutrients and gasses. The agitator 408 enhances the growth and productivity of the cultured organisms.

The reactor tank 400 further includes a port 410 that extends through the wall of the body 402 and provides a position to secure a sight glass 412 to the body 402. The port 410 provides a transition between the curved walls of the reactor tank 400 and the planar form factor of the sight glass 412. The sight glass 412 is planar to prevent optical distortion of the laser during spectrometry. The sight glass 412 is held in place with a frame 414 that defines holes for fasteners to secure the sight glass 412 to the port 410. The sight glass 412 is sandwiched between the frame 414 and the port 410 and the fasteners extend through the frame 414 and into the wall of the body 402 at the port 410. Additional components such as gaskets, seals, and other such components are included to ensure the sight glass 412 provides an air-tight seal and that no leakage occurs at the port 410.

The sight glass 412 is formed of an optically transparent material and is planar in form factor, to prevent distortion of light as it passes through the sight glass 412. In some examples the sight glass 412 may be formed of BK7 borosilicate glass and/or sapphire, or other such materials known for their optical transparency and unique Raman spectra.

The sight glass 412 may be located in-line with one of the mixing impellers of the agitator 408 within the reactor tank 400. The location, in-line with the mixing impeller may refer to a vertical as well as angular orientation and positioning of the port 410 and sight glass 412. The agitator 408 is disposed at a center of the reactor tank 400 with the mixing impeller positioned at a first height within the reactor tank 400. The sight glass 412 and mixing impeller are positioned such that an optical axis 416 that extends perpendicular to the sight glass 412 passes through the mixing impeller. This placement maximizes agitation at the sight glass location, reducing the potential for film buildup on the sight glass 412 and enhances the flow of biological media past the sight glass 412. This, in turn, contributes to increased homogeneity during bioprocessing, an important factor in maintaining product quality and yield as well as providing improved spectroscopy results during sampling. The sight glass 412 is further oriented such that the optical axis 416 (e.g., an axis perpendicular to the surface of the sight glass 412 and extending from a center of the sight glass 412 is directed towards a center of the reactor tank 400. In this manner, the optical axis 416 may align with a radius of the body 402.

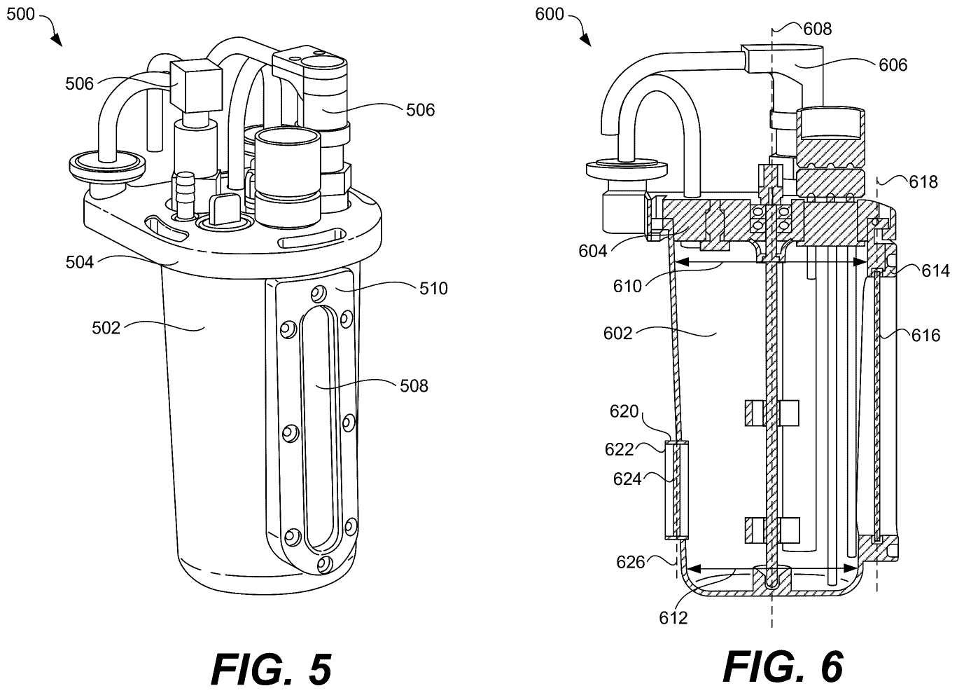

FIG. 5 illustrates a reactor tank 500 for a bioreactor system 100, according to an example. The reactor tank 500 may be similar or identical to the reactor tank 400 of FIG. 4 and/or the vessel 110 of FIGS. 1-3. The reactor tank 500 may be formed of a suitable material for containing a reaction, such as a polycarbonate, stainless steel, or other such rigid material that may be sterilized before introducing reactants. The reactor tank 500 is shown with a body 502 and a lid 504. The lid 504 includes connections 506 for sensors, gas, fluid, and sampling.

The reactor tank 500 further includes a viewing window 508 affixed within a frame 510. The viewing window 508 extends a substantial portion (e.g., greater than fifty percent) of the height of the reactor tank 500. The viewing window 508 provides for visibility into the reactor tank 500, for example for sensing systems such as spectrometry and/or for other inspection such as manual inspection. The height of the viewing window 508 allows visibility to observe any potential stratification within the reactor tank 500 and also enables sampling using optical means at varying heights within the reactor tank 500, for example if a bioreaction taking place segregates into disparate portions that separate vertically based on density, solubility, or otherwise separate. Similar to the sight glass 412 of FIG. 4, the viewing window 508 is held in place with a frame 510 that secures the viewing window against the wall of the body 502. In examples, the frame 510 also provides for orienting the viewing window 508 and providing a transition between the curved wall of the reactor tank 500 and the flat plane of the viewing window 508. Similar to the sight glass 412, the viewing window may be formed of a material such as borosilicate glass, sapphire, or other optically transparent material with a unique spectroscopic identity. Accordingly, the viewing window 508 provides for the optical probe described herein to be moved along a height of the reactor tank 500 for sampling.

FIG. 6 illustrates a section view of a reactor tank 600 for a bioreactor system 100, according to an example. The reactor tank 600 includes a body 602, lid 604, and connections 606 similar or identical to the body 502, lid 504, and connections 506 of FIG. 5. The reactor tank 600 is shown in section view with a central axis 608 that extends along a height of the reactor tank 600 through a center of the reactor tank 600. The central axis may align with an agitator, such as the agitator 408 of FIG. 4, which may rest in a feature at the bottom of the interior of the body 602 to maintain the agitator in the center of the reactor tank 600 along the center axis 608.

The body 602 has a tapered cylindrical shape, e.g., a frusto-conical shape, with a first diameter 610 adjacent an upper end or a top end of the body 602 and a second diameter 612 adjacent a lower end of the body 602. The tapered shape of the reactor tank 600 may aid with insertion into the bioreactor system 100 during assembly. However, the tapered shape may cause optical problems when attempting to use a spectrometer to determine the makeup of the contents through the wall of the body 602 when the body 602 has curved walls that are also pitched at an angle relative to the center axis 608.

To provide for consistent and accurate sampling using optical methods, such as spectrometry, a frame 614, similar or identical to the frame 510 of FIG. 5, is disposed on the body 602 and provides for supporting and orienting a viewing window 616 to enable sensing through the body 602 without, or with limited, optical distortion. The viewing window 616, which may be similar or identical to the viewing window 508 of FIG. 5, is oriented, as shown in the section view, such that an axis 618 extending along a height of the viewing window is parallel with the center axis 608. The axis 618 that defines the pose of the viewing window 616 is vertical with respect to gravity and provides for the viewing window 616 to be perpendicular to a horizontal surface such as a supporting surface.

At a second side of the reactor tank 600 the body 602 includes a port 620 with a frame 622 and sight glass 624 similar or identical to the sight glass 412 shown and described with respect to FIG. 4. The sight glass 624 may be similarly oriented vertically, with an axis 626 defining a vertical axis of the sight glass 624 parallel with the center axis 608. In examples, the reactor tank 600 may include the viewing window 616, the sight glass 624, or both. Accordingly, the reactor tank 600 may be configured with one or both of the viewing window 616 and the sight glass 624.

FIG. 7 illustrates a diagram of a bioreactor array 700 including multiple bioreactor systems 702A, 702B, 702C, 702D, 702E, 702F, 702G, 702H (collectively “bioreactor systems 702”) with a single optical emitter and sensor 712 to perform optical sensing of characteristics within the bioreactor systems 702, according to an example. The bioreactor systems 702 may be examples of the bioreactor system 100 described herein. The bioreactor array 700 further includes a linear rail 704, probe 706, and cart 708. The bioreactor array 700 is configured such that the bioreactor systems 702 are serviced and sensed by a single probe that moves between bioreactor systems 702. The probe 706 enables continuous acquisition of data and closed-loop control of the bioreactor systems 702 through the seamless integration of spectrometry. The bioreactor systems 702 are arranged adjacent one another, e.g., along a first direction parallel with the linear rail 704. Each of the bioreactor systems 702 is equipped with a reactor tank and each of the bioreactor systems 702 is equipped with a port for compatibility with the probe 706, such as the geometry of the housing 102 is shown and described with respect to FIGS. 3 and 8 herein, such that the port is aligned with the sight glass of the reactor tank to enable the probe 706 to capture data with respect to the internal environment of the reactor tank. In examples, the port may provide direct visibility from a probe 706 to the sight glass. In examples, the port may include an optical material that is used to direct the laser light from the probe into the sight glass and return the reflected light to the probe. Such elements may include mirrors and optically transparent materials to reflect and transport the light as needed to reach between the sight glass and the probe 706. The probe 706 is moved from one bioreactor system to the next to obtain data from samples during the length of an experiment, thereby eliminating the need for multiple probes for the bioreactor array 700 and thereby providing cost and aspace-saving measures.

The length of an experiment using bioreactor systems 702 may range significantly, such as for example from two to fourteen days. A datapoint may be collected by the probe 706 from each bioreactor system 702 at given intervals, such as for example with each bioreactor system being measured every fifteen minutes. The duration of each data point collection will vary depending on the nature of the media inside the reactor tank, such as for example for thirty seconds to five minutes; longer sampling intervals may be strategically chosen to maximize data accuracy while considering specific media properties. In examples, the intervals between sampling and the length of time for sampling each of the bioreactor systems 702 using the probe 706 may be determined and/or controlled based on control information from a central controller 714 that receives inputs related to the reactions occurring in each bioreactor and other information such as monitoring intervals, expected time periods for reactions to occur, and other such information. The central controller 714 may adjust, in some examples, the sampling rate for the bioreactor systems 702 based on the data obtained. For example, if a particular bioreactor system is reaching completion of a reaction sooner than the other bioreactor systems, the central controller may determine to increase the sampling rate of the particular bioreactor system until the reaction is determined to be completed or to have reached a predetermined threshold. The central controller 714 may also control operations of the bioreactor systems 702, for example to alter heating, cooling, and other systems of the bioreactor systems 702. To accomplish such control, the central controller may include one or more processors and one or more non-transitory computer-readable media having instructions stored thereon to cause the one or more processors to perform operations relating to control of the bioreactor systems 702, linear rail 704, probe 706, cart 708, emitter and sensor 712, or other system components.

Throughout the length of an experiment, the probe 706 may be precisely and rapidly moved from one bioreactor system to another at predetermined intervals as determined and/or controlled by the central controller 714. To facilitate the precise movement, the bioreactor system 702 may include probe-locating features. The probe locating features may include physical features such as physical stops, channels, grooves, and ridges to aid in positioning the probe 706. The probe locating features may also include visible or non-visible markets such as unique visual identifiers that may be detected by a camera system of a probe 706 and/or conveyor device such as the linear rail 704 and/or non-visual features such as conductive, capacitive, and/or magnetic sensors and systems to detect alignment of the probe with the bioreactor system. The probe-locating features are important in accurately positioning the probe in relation to the specialized port. These features enable probe placement with an accuracy level, such as for example within a first range of less than 0.5 millimeters in the X and Y directions and in a second range of less than 0.1 millimeters in the Z direction. In examples, the first range may be between zero and 1 millimeter and the second range may be between zero and 0.5 millimeters. The Z direction measurement defines the distance between the end of the probe and the surface of the sight glass or another port feature on the reactor tank. The placement of the probe is important for consistent measurements using the probe.

The probe 706 is mounted to a cart 708 that may include a motorized cart that smoothly traverses along the linear rail 704. Other linearly positionable systems such as conveyor systems, linear actuators, linear rails, and other such linearly positionable systems may be implemented to adjust a position of the probe 706 along a direction extending between the various bioreactor systems 702. The cart 708 may be motorized and is able to precisely locate the probe 706 with respect to the sight glass of the reactor tanks. The linearly positionable system (e.g., the linear rail 704 and cart 708 or other equivalent systems) may include a robotic system that facilitates the accurate positioning of the probe with respect to the sight glass or port of the bioreactor system, ensuring data accuracy and consistency between measurements. The three-dimensional position of the probe with respect to the sight glass may be adjusted by additional actuators to shift a position of the probe along the X, Y, or Z directions. In examples, the probe 706 may rest on a cart 708 that is linearly positionable along a first axis that extends across the multiple bioreactor systems 702. The position of the probe 706 with respect to directions perpendicular to the first axis may be fixed or positionable and controllable by servos or actuators that may be controlled by the central controller 714 to orient the probe 706 to a correct position for imaging, to ensure accuracy and consistency of measurement.

The probe 706 is connected to an emitter and sensor 712 that includes a spectrometer and laser source. The probe 706 may be connected to a Raman spectrometer and a laser source via a flexible cable 710 including fiber optics. For example, the flexible cable 710 may include two fiber optic cables, one for transmitting the laser from the laser source to be emitted at the probe 706 and the second for carrying light from the probe 706 to the spectrometer. The flexible cable 710 enables the probe 706 to move between the bioreactor systems 702 without requiring the entire spectrometer to be movable, and instead provides for only the probe 706 to be moved, which contributes to accuracy and consistency of positioning.

FIG. 8 illustrates a section view of a bioreactor system 800 interfacing with an optical probe 814 for spectroscopic analysis of the bioreactor tank 808, according to an example. The bioreactor system 800 is an example of the bioreactor system 100 described herein. The bioreactor system 800 includes a housing 802 and a lid 804. The housing 802 defines a port 806 providing optical access to a reactor tank 808 with a lid 810 secured within the bioreactor system 800. The reactor tank 808 may be similar or identical to other reactor tanks described herein and includes a sight glass 812 similar or identical to the sight glass 412 of FIG. 4.

The probe 814 connects to an emitter and sensor 712 such as shown and described with respect to FIG. 7. The probe 814 is movable between adjacent bioreactor systems 800 along a linear positioning system 816 such as the linear rail 704 of FIG. 7 (extending into and out of the page in FIG. 8).

The probe 814 may include a positioning system such as a sensor 818 that is configured to interact, along axis 820 with an alignment feature such as an optical marker or other sensing system to accurately position the probe 814 with respect to the port 806. In an example, the sensor 818 may include a camera and the axis 820 may be a viewing axis of the camera that aligns a visible marker at a particular location within the field of view of the camera by commands from a central controller, to accurately position the probe 814. In examples, the central controller instructs the probe 814 to reposition for gathering sensor data with respect to a particular bioreactor system. The central controller then receives sensor data from the sensor 818 and provides further instructions to the linear positioning system 816 to cause the probe 814 to be aligned with the port 806. In some examples the probe 814 may include additional actuators or components that the central controller may instruct to move the probe 814 vertically and/or horizontally as depicted in FIG. 8 for additional control of the placement of the probe 814 relative to the port 806. Such fine tuning may account for the bioreactor systems being on a somewhat uneven surface that results in varying heights of ports of respective bioreactor systems.

The probe 814 provides for emission and capture of laser light at an aperture 822 where laser from the laser source is emitted along axis 824 through the port 806 and the sight glass 812 to reach the interior of the reactor tank 808. In an eambodiment, the central controller instructs the probe 814 to emit the laser and capture light data at the aperture 822 (or other sensor of the probe 814) and convey the received light to the spectrometer for analysis.

Generally, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope or spirit of the present invention.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

What is claimed is:

1. A bioreactor comprising:
a reactor tank;
a liquid input line in fluid communication with the reactor tank to provide a liquid medium to an interior of the reactor tank;
a gas input line in fluid communication with the reactor tank to provide a gas to the interior of the reactor tank;
an agitator positioned in the reactor tank; and
a sight glass disposed on a wall of the reactor tank, the sight glass positioned at a first height from a bottom of the reactor tank, the first height lower than a second height indicative of a maximum fill height for the liquid medium within the reactor tank, and the sight glass adapted to interact with light-based measurement technologies.

2. The bioreactor of claim 1, wherein the sight glass includes a planar and optically transparent material.

3. The bioreactor of claim 1, wherein the sight glass includes sapphire.

4. The bioreactor of claim 1, wherein the sight glass includes borosilicate glass.

5. The bioreactor of claim 1, wherein:
the wall of the reactor tank includes a curved wall portion defining an opening; and
the sight glass includes a planar material secured to the curved wall portion at the opening, the sight glass secured by a frame connecting the planar material to the curved wall portion.

6. The bioreactor of claim 5, wherein the sight glass is positioned perpendicular to a radius of the reactor tank.

7. The bioreactor of claim 1, wherein the reactor tank tapers from a first diameter adjacent a top end to a second diameter adjacent a bottom end, the first diameter greater than the second diameter, and
wherein the sight glass is disposed vertically and perpendicular to an incident ray of the light-based measurement technologies.

8. A system comprising:
one or more bioreactors, each bioreactor of the one or more bioreactors including:
a reactor tank; and
a sight glass disposed on a wall of the reactor tank, the sight glass positioned at a first height from a bottom of the reactor tank, the first height lower than a second height indicative of a maximum fill height for a liquid medium within the reactor tank;
an optical probe configured to perform optical spectroscopy to determine sensor data associated with an environment within the reactor tank; and
a conveyance device configured to position the optical probe to align with the sight glass of the one or more bioreactors.

9. The system of claim 8, wherein the optical probe includes a Raman spectroscopy probe.

10. The system of claim 8, wherein:
the one or more bioreactors are arranged adjacent one another along a first axis; and
the conveyance device includes a track-based system configured to position the optical probe at positions along the first axis such that the optical probe aligns with respective sight glasses of the one or more bioreactors.

11. The system of claim 8, wherein the conveyance device is configured to maintain the optical probe perpendicular to the sight glass of the one or more bioreactors as the optical probe moves between the one or more bioreactors.

12. The system of claim 8, wherein the sight glass includes at least one of sapphire or borosilicate glass.

13. The system of claim 8, wherein the optical probe includes:
an optical source configured to produce light used for spectroscopic analysis of contents of the reactor tank;
a cable optically connected to the optical source and configured to transport the light from the optical source to an end of the cable and transport light to a spectrometer from the reactor tank; and
a cart disposed on the conveyance device and connected to the end of the cable, the cart configured to move between the one or more bioreactors for performing spectroscopy of the one or more bioreactors using the optical source.

14. The system of claim 8, further comprising one or more probe locating features disposed on respective bioreactors of the one or more bioreactors, the one or more probe locating features configured to guide positioning of the optical probe between the one or more bioreactors.

15. A bioreactor system comprising:
a first bioreactor;
a second bioreactor disposed adjacent the first bioreactor, the first bioreactor and the second bioreactor including, respectively:
a reactor tank, and
a sight glass disposed on a wall of the reactor tank, the sight glass positioned at a first height from a bottom of the reactor tank, the first height lower than a second height indicative of a maximum fill height for a liquid medium within the reactor tank;
an optical probe configured to perform optical spectroscopy to determine sensor data associated with an environment within the reactor tank; and
a conveyance device configured to position the optical probe to align with the sight glass of the first bioreactor and the second bioreactor.

16. The bioreactor system of claim 15, wherein the sight glass includes at least one of sapphire or borosilicate glass.

17. The bioreactor system of claim 15, wherein the optical probe includes:
a laser source;
a spectrometer; and
a fiber optic cable connected between the laser source, the spectrometer, and the conveyance device, the fiber optic cable configured to carry first light from the laser source to the reactor tank and carry second light to the spectrometer from the reactor tank.

18. The bioreactor system of claim 15, wherein the conveyance device includes a linear rail configured to move at least a portion of the optical probe along a first axis between the first bioreactor and the second bioreactor.

19. The bioreactor system of claim 15, wherein the first bioreactor and the second bioreactor include one or more probe locating features configured to interact with a component of the conveyance device and configured to guide positioning of the optical probe between the first bioreactor and the second bioreactor.

20. The bioreactor system of claim 15, wherein the conveyance device is configured to maintain the optical probe perpendicular to the sight glass of the first bioreactor and the sight glass of the second bioreactor as the optical probe moves between a first position and a second position.
